(19) United States
(12) Patent Application Publication
(10) Pub. No.: US 2022/0313898 A1
(43) Pub. Date: Oct. 6, 2022

(54) FLUID MANIFOLD ARRAY

(71) Applicant: CareFusion 303, Inc., San Diego, CA (US)

(72) Inventors: Wesley Underwood, Placentia, CA (US); George Mansour, Diamond Bar, CA (US)

(21) Appl. No.: 17/710,688

(22) Filed: Mar. 31, 2022

Related U.S. Application Data
(60) Provisional application No. 63/171,337, filed on Apr. 6, 2021.

(51) Int. Cl. A61M 5/14

(52) U.S. Cl. CPC A61M 5/1408

(57) ABSTRACT

Fluid manifolds are described herein. A fluid manifold includes a manifold body and a valve. The manifold body defines an inlet port, an outlet port, a flow channel, and a divider volume. The flow channel is in fluid communication with the inlet port and the outlet port. The divider volume is in fluid communication with the injection port and the flow channel. The valve includes a divider body. The divider body is movable within the divider volume. The divider body defines a divider port. The divider body can be moved to restrict or prevent flow from the injection port to the flow channel in a first position. The divider port can permit flow from the injection port to the flow channel in the second position of the divider body.

### REFERENCE TO RELATED APPLICATIONS

This application claims priority to U.S. Provisional Application No. 63/171,337, filed Apr. 6, 2021, the entire disclosure of which application being incorporated herein by this reference.

### FIELD OF THE INVENTION

The present disclosure generally relates to medication delivery systems and, in particular, to fluid manifolds.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid. During some medical treatments, more than one medical fluid is delivered to the patient simultaneously. For example, one or more drugs can be introduced into the infusate that is administered to the patient.

In some applications, the administration of multiple medical fluids may be a complex procedure and may result in imprecise administration of certain medical fluids.

### SUMMARY

The disclosed subject matter relates to fluid manifolds. In certain embodiments, a fluid manifold is disclosed that comprises a manifold body defining: an inlet port; an outlet port; a flow channel in fluid communication with the inlet port and the outlet port; an injection port; and a divider volume in fluid communication with the injection port and the flow channel; and a valve comprising a divider body movable between a first position and a second position within the divider volume, the divider body defining a divider port, wherein the divider body restricts or prevents flow from the injection port to the flow channel in the first position and the divider port permits flow from the injection port to the flow channel in the second position.

In certain embodiments, a fluid manifold array is disclosed that comprises a first fluid manifold comprising: a manifold body defining: an inlet port; an outlet port; a flow channel in fluid communication with the inlet port and the outlet port; an injection port; and a divider volume in fluid communication with the injection port and the flow channel; and a valve comprising a divider body movable between a first position and a second position within the divider volume, the divider body defining a divider port, wherein the divider body restricts or prevents flow from the injection port to the flow channel in the first position and the divider port permits flow from the injection port to the flow channel in the second position; and a second fluid manifold comprising a second inlet port and a second outlet port in fluid communication with the second inlet port, wherein the second inlet port is coupled to the outlet port of the first fluid manifold, and the inlet port of the first fluid manifold is in fluid communication with the second outlet port. In certain embodiments, a method to control flow through a manifold is disclosed that comprises permitting flow from an inlet port to an outlet port via a flow channel; introducing a syringe into an injection port, wherein the injection port is in fluid communication with a divider volume, the divider volume being in fluid communication with the flow channel; restricting or preventing flow from the injection port to the flow channel via a divider body disposed within the divider volume; and moving the divider body to permit flow from the injection port to the flow channel.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a perspective view of a fluid manifold array, in accordance with various aspects of the present disclosure.

FIG. 2 is a cross-sectional view of a fluid manifold of the fluid manifold array of FIG. 1 in a resting position.

FIG. 3 is a cross-sectional view of a fluid manifold of the fluid manifold array of FIG. 1 in a depressed position.

FIG. 4 is an exploded view of a fluid manifold of the fluid manifold array of FIG. 1.

### DETAILED DESCRIPTION

The disclosed fluid manifold incorporates a valve to control flow from an injection port into the flow channel of the manifold. The valve includes a divider body that is movable within a divider volume to control flow into the flow channel. By utilizing a divider body within a divider volume, the manifold can reduce the “dead volume” of residual medications remaining in the injection port after administration of the medication.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the administration of medical fluid using the disclosed manifold, it is to be understood that this description is only an example of usage and does not limit the scope of the claims. Various aspects of the disclosed manifold and manifold array may be used in any application where it is desirable to provide for the administration of medical fluids

The disclosed manifold overcomes several challenges discovered with respect to certain conventional manifolds. One challenge with certain conventional manifolds is that conventional manifolds may have large diameter channels leading to the infusate stream and therefore have a large dead volume where different drug residuals may mix. Further, certain components, such as the channel within the stopcock cannot be flushed. Additionally, certain conventional manifolds can have open ports, which are exposed to the environment when syringes are changed or removed, potentially contaminating the fluid stream. Further, conventional manifolds that utilize twisting stopcocks require the clinician to remember to twist the stopcock to open before administering the drug and then remember to twist the stop cock closed. Because mixing of residual drugs and fluid contamination may have adverse effects on the patient and clinicians may forget to open or close a stopcock, leading to backflow into a syringe, the use of conventional manifolds is undesirable.

Therefore, in accordance with the present disclosure, it is advantageous to provide a fluid manifold as described that reduces the mixing of residual drugs by reducing the dead space of the manifold, avoids fluid contamination by reducing the area exposed to the environment, and isolates the infusate stream from the connected syringes.

An example of a fluid manifold that reduces the mixing of residual drugs, avoids fluid contamination, and isolates the infusate stream is now described.

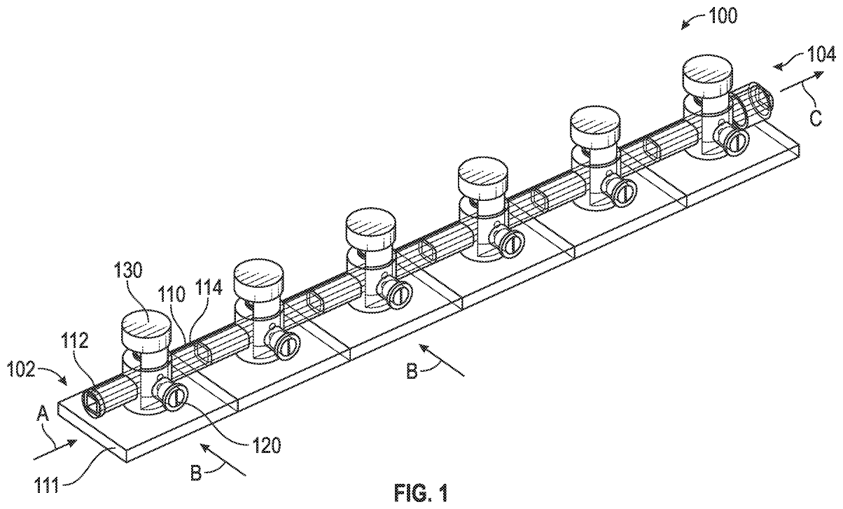

FIG. 1 is a perspective view of a fluid manifold array 100, in accordance with various aspects of the present disclosure. In the depicted example, the fluid manifold array 100 allows infusate to flow through the fluid manifold array 100.

As illustrated, an infusate stream A or any other stream of medical fluid (e.g. a saline solution, liquid medication, or any combination thereof) can flow through an inlet port 102, through the fluid manifold array 100, and through the outlet port 104 to administer the infusate stream C to the patient. In the depicted example, the fluid manifold array 100 can be assembled or formed from one or more fluid manifolds 110 that are joined or otherwise coupled together. Therefore, the infusate stream A can flow through each of the fluid manifolds 110 forming the fluid manifold array 100 to administer the infusate stream C to the patient.

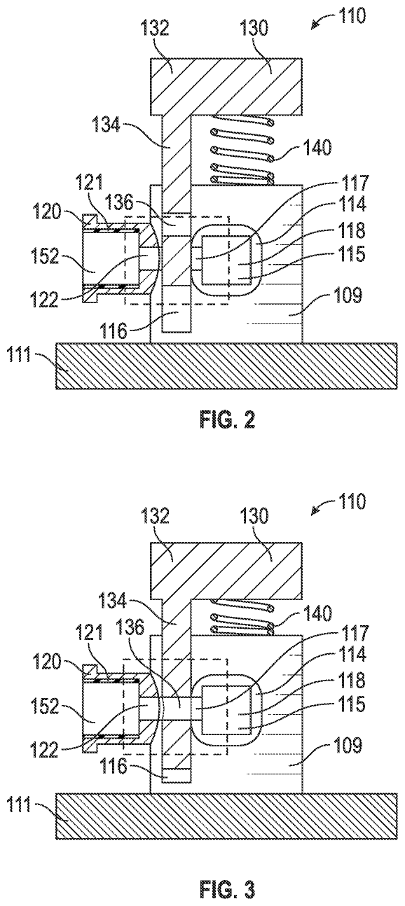

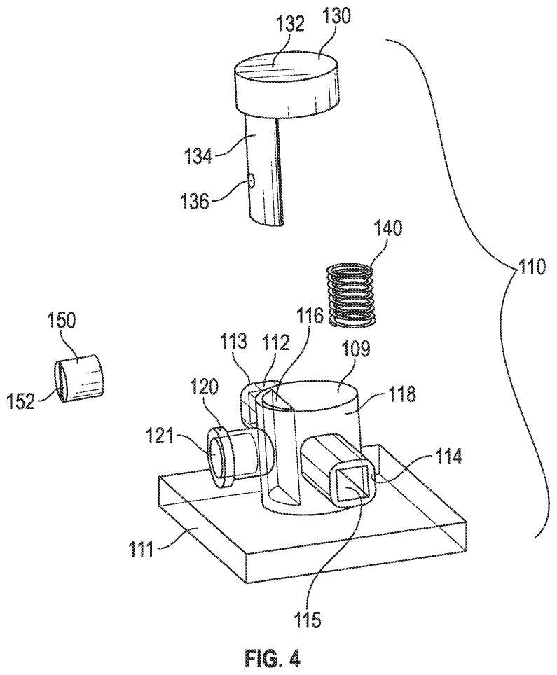

FIG. 2 is a cross-sectional view of a fluid manifold 110 of the fluid manifold array 100 of FIG. 1 in a resting position. FIG. 3 is a cross-sectional view of a fluid manifold 110 of the fluid manifold array 100 of FIG. 1 in a depressed position. FIG. 4 is an exploded view of a fluid manifold 110 of the fluid manifold array 100 of FIG. 1. With reference to FIGS. 1-4, in some embodiments, each fluid manifold 110 includes a body 109 that defines an inlet port 112 and an outlet port 114. As illustrated, the body 109 can further define a flow channel 118 that allows flow received from the inlet lumen 113 of the inlet port 112 to flow through the flow channel 118 to the outlet lumen 115 of the outlet port 114.

As illustrated in FIG. 1, multiple fluid manifolds 110 can be coupled or daisy-chained together to provide a desired fluid manifold array 100. As can be appreciated, any suitable number of fluid manifolds 110 can be coupled together to provide a desired configuration for the fluid manifold array 100. Optionally, the body 109 of the fluid manifold 110 can define a base portion 111 to facilitate mounting and/or coupling of the fluid manifolds 110 to each other or to a common mounting plate or base.

In the depicted example, an outlet port 114 of a fluid manifold 110 can be coupled to an inlet port 112 of an adjacent fluid manifold 110 to permit flow to be transferred from one fluid manifold 110 to another fluid manifold 110 within the fluid manifold array 100. As can be appreciated, the inlet port 112 of the first fluid manifold 110 can serve as the inlet port 102 of the fluid manifold array 100 and the outlet port 114 of the last fluid manifold 110 can serve as the outlet port 104 of the fluid manifold array 100. Accordingly, the infusate stream A received by the inlet port 102 of the fluid manifold array 100 can pass through the inlet port 112, the flow channel 118, and the outlet port 114 of each fluid manifold 110 of the fluid manifold array 100, and through the outlet port 104 of the fluid manifold array 100 to administer the infusate stream C to the patient.

Any of the ports described below may include any number of sealing devices or configurations. Such sealing devices or configurations may be adapted to allow various devices to be inserted therethrough while also providing a fluid seal around an inserted device.

As can be appreciated, a wide variety of materials may be used in the construction of embodiments such as those disclosed herein. Typical materials may include a polycarbonate for the body 109 of the fluid manifold 110, though any suitable material including, for example, acrylics, polyolefins, engineering polymers, blends and alloys of any of an array of polymers, as well as self-lubricating polymers may be used. Sealing may be accomplished by the use of an interference fit, though alternative methods may be used including the use of elastomeric seals and o-rings. The various components may be assembled using bonding techniques including solvents, UV cured adhesives; welding techniques including ultrasonic, thermal or spin techniques; or snap fits and/or other mechanical attachment devices.

During operation, the fluid manifolds 110 of the fluid manifold array 100 allow for the introduction of a drug stream B or any other stream of medical fluid to be combined with the infusate stream A to be administered to the patient as the combined infusate stream C. In the depicted example, each fluid manifold 110 can allow for a drug stream B to be combined with the infusate stream A flowing therethrough.

As illustrated, each fluid manifold 110 includes an injection port 120 to allow for the introduction of drug stream B into the flow channel 118. In some applications, the injection port 120 can receive a drug stream B from a syringe or other fluid source coupled to the injection port 120. The injection port 120 can define an injection port lumen 121 that allows for flow to be directed toward the flow channel 118. As can be appreciated, the fluid manifold array 100 can include any suitable number of fluid manifolds 110 to provide a desired number of injection ports 120.

Optionally, the injection port 120 can include a sealing member 150 disposed within the injection port lumen 121. The sealing member 150 can seal against the injection port lumen 121 to reduce the exposure of the flow channel 118 and the infusate stream A to the environment, reducing the chance of contamination and/or blood stream infections through the infusate stream C.

During operation, the sealing member 150 can permit the introduction of a syringe or other fluid source into the injection port 120. The sealing member 150 can define a split septum 152 to permit the syringe or other fluid source to be inserted past the sealing member 150. The split septum 152 can seal against the syringe or other fluid source to minimize exposure of the infusate stream A to the environment.

As described herein, the injection port 120 can provide fluid communication between the injection port lumen 121 and the flow channel 118. As illustrated in FIGS. 2 and 3, the flow can pass from the injection port lumen 121 through a divider volume 116 and into the flow channel 118. The divider volume 116 can be defined within the body 109 between the injection port lumen 121 and the flow channel 118.

In some embodiments, fluid flow from the injection port lumen 121 can be introduced into the divider volume 116 via a first injection channel 122. The first injection channel 122 is defined in the body 109 and provides fluid communication between the injection port lumen 121 and the divider volume 116. Fluid flow from the divider volume 116 can be introduced into the flow channel 118 via a second injection channel 117. The second injection channel 117 is defined in the body 109 and provides fluid communication between the divider volume 116 and the flow channel 118.

Therefore, flow from the injection port lumen 121 can flow from the first injection channel 122 to the second injection channel 117 across the divider volume 116. As illustrated, the first injection channel 122 and the second injection channel 117 can be disposed on opposite sides of the divider volume 116. Optionally, the first injection channel 122 and the second injection channel 117 can be aligned within the divider volume 116.

In the depicted example, a valve 130 can control flow from the injection port 120 into the flow channel 118. As illustrated, the valve 130 can include a divider body 134 that is movable within the divider volume 116 to control the flow between the injection port 120 and the flow channel 118. As can be appreciated, the configuration of the divider body 134 within the divider volume 116 can reduce or minimize the dead volume within the divider volume 116, minimizing the mixing of residual drugs introduced into the flow channel 118.

As illustrated in FIG. 2, the valve 130 can restrict or prevent flow from the injection port 120 into the flow channel 118 in a resting or sealing position. As illustrated, in the divider body 134 can block flow from the injection port 120 to the flow channel 118 by covering, sealing, or otherwise obscuring the flow path between the first injection channel 122 and the second injection channel 117. In some embodiments, the divider body 134 restricts or prevents flow from the injection port 120 from entering the divider volume 116, restricting or preventing any residual drugs or fluid from remaining in the divider volume 116.

Optionally, the valve 130 can include a biasing member 140 to urge the valve 130 into the resting or sealing position. During operation, when the valve 130 is not actuated, the biasing member 140 can urge the divider body 134 to the resting or sealing position to restrict or prevent flow between the injection port 120 and the flow channel 118. The biasing member 140 can be a spring or other urging member. Advantageously, the biasing member 140 can restrict or prevent unintentional drug mixing or backflow from a drug or medical fluid being administered in an adjacent injection port 120 by sealing the injection port 120 from the flow channel 118 when the injection port 120 is not in use.

As shown in FIG. 3, the valve 130 can be depressed, actuated, or otherwise moved into an actuated or flow position to allow flow from the injection port 120 into the flow channel 118. In the depicted example, the divider body 134 can define a divider port 136 that allows flow therethrough. Therefore, as illustrated, the divider body 134 can be moved to permit flow from the injection port 120 to the flow channel 118 via the divider port 136. In some embodiments, the divider port 136 is aligned to create a flow path between the first injection channel 122, the divider port 136, and the second injection channel 117. As can be appreciated, the divider body 134 can be moved within the divider volume 116 to permit the divider port 136 to be aligned with the first injection channel 122 and the second injection channel 117 in the flow position. Advantageously, the configuration of the divider port 136 within the divider volume 116 minimizes the dead volume of residual drugs within the fluid manifold 110.

Optionally, the valve 130 can include a button 132 to allow a clinician to move the divider body 134 into the flow position. During operation, the valve 130 can be actuated by pressing the button 132, allowing the clinician to dispense the drug or medical fluid into the flow channel 118 via the injection port 120. During actuation of the valve 130 the biasing member 140 can be compressed or otherwise energized.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A fluid manifold including: a manifold body defining: an inlet port; an outlet port; a flow channel in fluid communication with the inlet port and the outlet port; an injection port; and a divider volume in fluid communication with the injection port and the flow channel; and a valve including a divider body movable between a first position and a second position within the divider volume, the divider body defining a divider port, wherein the divider body restricts flow from the injection port to the flow channel in the first position and the divider port permits flow from the injection port to the flow channel in the second position.

Clause 2. The fluid manifold of Clause 1, wherein the manifold body further defines a first injection channel in fluid communication with the injection port and the divider volume.

Clause 3. The fluid manifold of Clause 2, wherein the manifold body further defines a second injection channel in fluid communication with the divider volume and the flow channel.

Clause 4. The fluid manifold of Clause 3, wherein the divider port permits flow from the first injection channel to the second injection channel in the second position.

Clause 5. The fluid manifold of Clause 3, wherein the first injection channel and the second injection channel are aligned.

Clause 6. The fluid manifold of Clause 5, wherein the divider port is aligned with the first injection channel and the second injection channel in the second position.

Clause 7. The fluid manifold of Clause 3, wherein the divider body prevents flow from the first injection channel to the second injection channel in the first position.

Clause 8. The fluid manifold of Clause 1, wherein the valve comprises a button coupled to the divider body, and the button is configured to move the divider body between the first position and the second position.

Clause 9. The fluid manifold of Clause 1, wherein the valve comprises a biasing member configured to urge the divider body toward the first position.

Clause 10. The fluid manifold of Clause 1, further including a sealing member disposed within the injection port, wherein the sealing member defines a split septum configured to receive a syringe.

Clause 11. A fluid manifold array, including: a first fluid manifold including: a manifold body defining: an inlet port; an outlet port; a flow channel in fluid communication with the inlet port and the outlet port; an injection port; and a divider volume in fluid communication with the injection port and the flow channel; and a valve including a divider body movable between a first position and a second position within the divider volume, the divider body defining a divider port, wherein the divider body restricts flow from the injection port to the flow channel in the first position and the divider port permits flow from the injection port to the flow channel in the second position; and a second fluid manifold including a second inlet port and a second outlet port in fluid communication with the second inlet port, wherein the second inlet port is coupled to the outlet port of the first fluid manifold, and the inlet port of the first fluid manifold is in fluid communication with the second outlet port.

Clause 12. The fluid manifold array of Clause 11, wherein the manifold body further defines a first injection channel in fluid communication with the injection port and the divider volume.

Clause 13. The fluid manifold array of Clause 12, wherein the manifold body further defines a second injection channel in fluid communication with the divider volume and the flow channel.

Clause 14. The fluid manifold array of Clause 11, wherein the valve comprises a button coupled to the divider body, and the button is configured to move the divider body between the first position and the second position.

Clause 15. The fluid manifold array of Clause 11, wherein the valve comprises a biasing member configured to urge the divider body toward the first position.

Clause 16. The fluid manifold array of Clause 11, further including a sealing member disposed within the injection port, wherein the sealing member defines a split septum configured to receive a syringe.

Clause 17. A method to control flow through a manifold, the method including: permitting flow from an inlet port to an outlet port via a flow channel; introducing a syringe into an injection port, wherein the injection port is in fluid communication with a divider volume, the divider volume being in fluid communication with the flow channel; restricting flow from the injection port to the flow channel via a divider body disposed within the divider volume; and moving the divider body to permit flow from the injection port to the flow channel.

Clause 18. The method of Clause 17, further including: aligning a divider port defined in the divider body with a first injection channel and a second injection channel to permit flow from the injection port to the flow channel, wherein the first injection channel is in fluid communication with the injection port and the divider volume and the second injection channel is in fluid communication with the divider volume and the flow channel.

Clause 19. The method of Clause 18, further including: misaligning the divider port to prevent flow from the injection port to the flow channel.

Clause 20. The method of Clause 17, further including: depressing a button coupled to the divider body to move the divider body to permit flow from the injection port to the flow channel.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid manifold comprising:
a manifold body defining:
an inlet port;
an outlet port;
a flow channel in fluid communication with the inlet port and the outlet port;
an injection port; and
a divider volume in fluid communication with the injection port and the flow channel; and
a valve comprising a divider body movable between a first position and a second position within the divider volume, the divider body defining a divider port, wherein the divider body restricts flow from the injection port to the flow channel in the first position and the divider port permits flow from the injection port to the flow channel in the second position.

2. The fluid manifold of claim 1, wherein the manifold body further defines a first injection channel in fluid communication with the injection port and the divider volume.

3. The fluid manifold of claim 2, wherein the manifold body further defines a second injection channel in fluid communication with the divider volume and the flow channel.

4. The fluid manifold of claim 3, wherein the divider port permits flow from the first injection channel to the second injection channel in the second position.

5. The fluid manifold of claim 3, wherein the first injection channel and the second injection channel are aligned.

6. The fluid manifold of claim 5, wherein the divider port is aligned with the first injection channel and the second injection channel in the second position.

7. The fluid manifold of claim 3, wherein the divider body prevents flow from the first injection channel to the second injection channel in the first position.

8. The fluid manifold of claim 1, wherein the valve comprises a button coupled to the divider body, and the button is configured to move the divider body between the first position and the second position.

9. The fluid manifold of claim 1, wherein the valve comprises a biasing member configured to urge the divider body toward the first position.

10. The fluid manifold of claim 1, further comprising a sealing member disposed within the injection port, wherein the sealing member defines a split septum configured to receive a syringe.

11. A fluid manifold array, comprising:
a first fluid manifold comprising:
a manifold body defining:
an inlet port;
an outlet port;
a flow channel in fluid communication with the inlet port and the outlet port;
an injection port; and
a divider volume in fluid communication with the injection port and the flow channel; and
a valve comprising a divider body movable between a first position and a second position within the divider volume, the divider body defining a divider port, wherein the divider body restricts flow from the injection port to the flow channel in the first position and the divider port permits flow from the injection port to the flow channel in the second position; and
a second fluid manifold comprising a second inlet port and a second outlet port in fluid communication with the second inlet port, wherein the second inlet port is coupled to the outlet port of the first fluid manifold, and the inlet port of the first fluid manifold is in fluid communication with the second outlet port.

12. The fluid manifold array of claim 11, wherein the manifold body further defines a first injection channel in fluid communication with the injection port and the divider volume.

13. The fluid manifold array of claim 12, wherein the manifold body further defines a second injection channel in fluid communication with the divider volume and the flow channel.

14. The fluid manifold array of claim 11, wherein the valve comprises a button coupled to the divider body, and the button is configured to move the divider body between the first position and the second position.

15. The fluid manifold array of claim 11, wherein the valve comprises a biasing member configured to urge the divider body toward the first position.

16. The fluid manifold array of claim 11, further comprising a sealing member disposed within the injection port, wherein the sealing member defines a split septum configured to receive a syringe.

17. A method to control flow through a manifold, the method comprising:
permitting flow from an inlet port to an outlet port via a flow channel;
introducing a syringe into an injection port, wherein the injection port is in fluid communication with a divider volume, the divider volume being in fluid communication with the flow channel;
restricting flow from the injection port to the flow channel via a divider body disposed within the divider volume; and
moving the divider body to permit flow from the injection port to the flow channel.

18. The method of claim 17, further comprising:
aligning a divider port defined in the divider body with a first injection channel and a second injection channel to permit flow from the injection port to the flow channel, wherein the first injection channel is in fluid communication with the injection port and the divider volume and the second injection channel is in fluid communication with the divider volume and the flow channel.

19. The method of claim 18, further comprising:
misaligning the divider port to prevent flow from the injection port to the flow channel.

20. The method of claim 17, further comprising:
depressing a button coupled to the divider body to move the divider body to permit flow from the injection port to the flow channel.
